(19) United States
(12) Patent Application Publication
(10) Pub. No.: US 2010/0111883 A1
(43) Pub. Date: May 6, 2010

(54) CYCLODEXTRIN FORMULATIONS

(76) Inventors: Peter Vitins, Zug (CH); Marcel Langenauer, Lauperdorf (CH)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 12/523,439

(22) PCT Filed: Jan. 17, 2008
(86) PCT No.: PCT/EP2008/000351

§ 371 Date: Dec. 30, 2009

Related U.S. Application Data
(60) Provisional application No. 60/885,293, filed on Jan. 17, 2007.

(51) Int. Cl. A61K 31/715; A61Q 17/04

(52) U.S. Cl. 424/59; 514/58

(57) ABSTRACT

The present invention is directed to various formulations comprising one or more cyclodextrins, said formulations being particularly useful to treating or preventing viral infections. In specific embodiments, the formulations comprise non-water based gels, throat and nose sprays, nose gels, lip balms, and emulsions.

### FIELD OF THE INVENTION

The invention relates to compositions comprising cyclodextrins. More particularly, the invention relates to pharmaceutical and cosmetic compositions including one or more cyclodextrin compounds.

### BACKGROUND

Medications and vaccines for treatment and prevention of infectious diseases have long been known. While many effective medications for bacterial infections have been developed over the years, it is only recently that effective treatments or prophylactics for viral infections have been found.

Most antiviral medications available today are targeted to viruses that cause sexually transmitted diseases (STDs). Non-limiting examples of STDs caused by infectious viral agents include genital herpes (caused by herpes simplex viruses, or HSVs); AIDS (caused by human immunodeficiency virus, or HIV); genital warts (caused by human papillomaviruses, or HPVs), spastic paralysis and adult T cell leukemia (caused by human T-cell leukemia or lymphotropic virus type 1 (HTLV-1)), and viral hepatitis (caused by hepatitis viruses, such as hepatitis B virus (HBV) and hepatitis C virus (HCV).

It is estimated that over 4 billion U.S. dollars per year are spent worldwide on the various pharmaceuticals prescribed to treat STDs arising from infectious viral agents. For example, famciclovir, acyclovir, penciclovir, valacyclovir, and foscarnet are all drugs prescribed for treatment of HSV-related diseases. These agents have been shown to speed the healing and the resolution of symptoms in both primary and recurrent episodes of genital herpes; however, the clinical use of acyclovir (also known as ACV), the current “gold standard” of anti-herpes medications, is limited. Moreover, many side effects are associated with these anti-viral agents, such as nausea, diarrhea, and headache. For example, foscarnet, when administered intravenously, can have several toxic effects, such as reversible impairment of kidney function or induction of seizures. Moreover, these drugs do not cure the herpes infection, but rather suppress the symptoms of the disease by inhibiting active replication of the virus.

Cyclodextrins, while not falling into the traditional antiviral categories, have been found effective at treating and preventing diseases caused by infectious viral agents. In light of the continued need for effective antiviral agents and the availability of non-traditional antivirals, it would be useful to have cosmetic or pharmaceutical compositions capable of treating or preventing viral infections. More particularly, it would be useful to have compositions effective for treatment or prevention of STDs, such as HSV-related diseases. Still further, it would be useful to have antiviral compositions provided in specific formulations that specifically target the areas associated with “breakouts” of various viral conditions and that are also convenient and discrete.

### SUMMARY OF THE INVENTION

The present invention provides pharmaceutical and cosmetic compositions comprising cyclodextrins such that the compositions are particularly useful for treating and preventing viral infections. The compositions are provided in a variety of formulations allowing convenient and discrete administration of the compositions.

The compositions of the invention particularly can be used to treat STDs. For example, the compositions can be used to treat infections caused by a wild-type herpes virus, such as HSV-1 or HSV-2, as well as drug resistant herpes viruses (e.g., acyclovir-resistant herpes viruses). In addition, the compositions can be used to treat infections caused by Epstein-Barr Virus (EBV), human papillomavirus (HPV), hepatitis virus, such as HBV or HCV, cytomegalovirus, molluscum contagiosum virus, or a pox virus (e.g., vaccinia). The inventive compositions may be used in the treatment of primary viral infections or recurrent viral infections and can thus be applied to a topical site where a primary viral infection actively exists or a site known to exhibit a recurrent viral infection. In one embodiment of the invention, the compositions are particularly useful against viral infections caused by a herpes virus. In another embodiment, the compositions are particularly useful against viral infections caused by a pox virus.

As noted above, the compositions of the invention generally comprise one or more cyclodextrins effective against viral infections, and such cyclodextrins may comprise the only active ingredient provided in the composition. In further embodiments, however, the compositions may comprise one or more additional antiviral drugs. Such additional antiviral drugs may enhance the therapeutic effect of the cyclodextrins or may simply act in addition to the effective action of the cyclodextrin. Non-limiting examples of additional antivirals that may be included in the compositions include famciclovir, acyclovir, valaciclovir, foscamet, and penciclovir. In one embodiment of the invention, the inventive compositions additionally comprise acyclovir.

The compositions of the invention generally comprise one or more cyclodextrins in an amount of about 0.1% to about 50% by weight, based on the overall weight of the composition. The cyclodextrin composition can vary within that range depending upon the type of composition and the intended use of the composition.

In addition to antivirals, such as disclosed above, the compositions of the invention may further include other agents effective in the treatment or prophylaxis of viral infections. For example, the compositions may include anti-retroviral agents, such as nucleoside analogue reverse transcriptase inhibitors, non-nucleoside analogue reverse transcriptase inhibitors, or protease inhibitors.

In certain aspects, the invention provides pharmaceutical or cosmetic compositions for application to a site where a primary viral infection actively exists or a site known to exhibit a recurrent viral infection. In specific embodiments, the composition comprises 0.1% to 50% by weight of a cyclodextrin or derivative thereof and a pharmaceutically or cosmetically acceptable excipient suitable such that the pharmaceutical or cosmetic composition is in the form of a non water-based gel, a nose or throat spray, a hydrogel suitable for application to mucus membranes, or a lip balm. Particularly, the pharmaceutically or cosmetically acceptable excipient can be selected from the group consisting of polyethylene glycol-based components, polyols, electrolytes, and waxy components. In certain embodiments, the cyclodextrin is the only agent present in the composition having antiviral activity or is not in the form of an inclusion complex with a further compound having antiviral activity.

In one aspect, the invention provides a gel composition. Such compositions are preferably in a form useful for topical administration to an area actively exhibiting signs of a viral infection. For example, the gel could be in a form useful for administration to a mouth sore indicative of an HSV infection (e.g., a “cold-sore”).

In one particular embodiment, the invention provides a non-water based gel composition comprising one or more cyclodextrins or salts or derivatives thereof. Preferably, the composition further comprises one or more polyethylene glycol-based components. Such gels according to the invention can be characterized in that the gel is a non water-based gel. In other words, the gel is substantially free of water. In further embodiments, the gel is also preservative-free.

In another aspect, the invention provides compositions that are particularly adapted for administration to the nose or throat. For example, in certain embodiment, the invention provides a sprayable composition. Such sprayable compositions are deliverable to nasal passages by spraying the composition directly into one or both nostrils or are deliverable to the throat by spraying into the mouth, particularly by-passing the anterior portions of the mouth and applying directly to the throat area. In one particular embodiment, the invention provides a sprayable composition comprising one or more cyclodextrins or salts or derivatives thereof and water. The sprayable composition can further comprise one or more polyols.

In further embodiments according to this aspect of the invention, there are provided nose gel compositions. Such nose gels can be distinguished from the gels for topical administration described above in that that nose gel compositions are formulated for direct contact with mucous membranes. In particular, the nose gel compositions of the invention preferably comprise one or more carriers or solvents that are non-irritating or are soothing to mucous membranes, such as in the nose of mammals. In one particular embodiment, a nose gel composition according to the invention comprises one or more cyclodextrins or salts or derivatives thereof, a carrier, and a thickener.

In yet another aspect, the invention provides cosmetic compositions that are particularly adapted for topical application to portions of the skin of a mammal prone to infection or actively exhibiting symptoms of a viral infection. For example, in certain embodiments, the invention provides lip balm compositions.

In one particular embodiment, the invention provides a lip balm composition comprising one or more cyclodextrins or salts or derivatives thereof, and one or more waxy components. In further embodiments, the lip balms can comprise additional beneficial components, such as one or more UV filter components.

### DETAILED DESCRIPTION OF THE INVENTION

The present inventions now will be described more fully hereinafter with reference to specific embodiments of the invention. Indeed, the invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

The present invention provides pharmaceutical and cosmetic compositions that are formulation for application to sites exhibiting an active viral infection (e.g., a cold sore or other physical symptom of a “breakout” indicative of an active viral infection) or to a site where there are no signs of an active infection but where recurrent breakouts are known to occur. As such, the compositions are useful for treating viral infections (i.e., reducing the duration of an active breakout associated with a viral infection) and preventing viral infections (e.g., preventing recurrent breakouts). The compositions are particularly useful for topical administration, such as to sore indicative of a viral infection. The term “topical administration” is used in its broadest sense to include administration to a surface on the body that is generally open to the surroundings. This includes not only the skin but also the nasal and oral passages. Thus, topical administration can include application to the skin, application to the nasal passages, and application to the oral cavity (including the upper throat). The compositions of the invention include a cyclodextrin and a pharmaceutically or cosmetically acceptable excipient suitable such that the pharmaceutical or cosmetic composition is in a form for administration to sites as described above.

Cyclodextrins (originally called cellulosine when first identified in the late 19^{th} Century and now sometimes called cycloamyloses) make up a family of cyclic oligosaccharides composed of 5 or more α-D-glucopyranoside units linked by α-(1,4) glycosidic linkages, as in amylose (a fragment of starch). The smallest (and non-naturally occurring cyclodextrin) is the 5-membered macrocycle. The largest, well-characterized cyclodextrin contains 32 1,4-anhydroglucopyranoside units, but at least 150-membered cyclic oligosaccharides are also known (although generally as a poorly characterized mixture).

The most commonly known cyclodextrins contain a number of glucose monomers ranging from six to eight units in a ring. The three naturally occurring cyclodextrins are six, seven, and eight sugar ring molecules typically known as α-cyclodextrin, β-cyclodextrin, and γ-cyclodextrin, respectively. For representative purposes, the chemical structure for β-cyclodextrin is provided below in Formula (I).

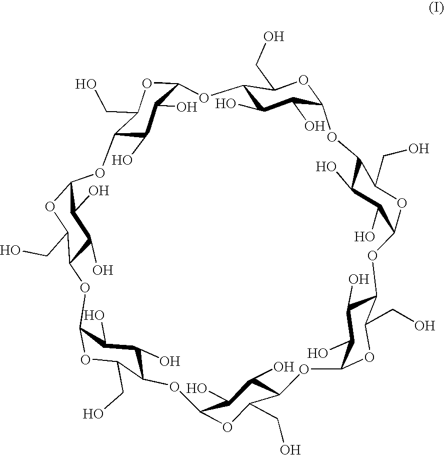

The most stable three dimensional molecular configuration for cyclodextrins in a solvent takes the form of a toroid with the upper (larger) and lower (smaller) opening of the toroid presenting secondary and primary hydroxyl groups, respectively, to the solvent environment. The interior of the toroid is hydrophobic as a result of the electron rich environment provided in large part by the glycosidic oxygen atoms. Early research indicated cyclodextrins formed stable, aqueous complexes with many compounds, and it is the interplay of atomic (Van der Waals), thermodynamic (hydrogen bonding), and solvent (hydrophobic) forces that is typically believed to account for the stable complexes that may be formed with chemical substances while in the apolar environment of the cyclodextrin cavity. In light of this complexing function, cyclodextrins have previously been used in combination with drugs to enhance solubility of poorly soluble drugs, form a drug-protective micro-environment, create and maintain stable homogeneous distributions, provide more convenient physical forms (e.g., suspension to solution or oil to solid), and alter their physical properties (e.g., smell and taste). Cyclodextrins are further generally described in Comprehensive Supramolecular Chemistry, Volume 3, Cyclodextrins (Lehn, Jean-Marie and Osa, Tetsuo, editors), Elsevier Science, Inc., which is incorporated herein by reference in its entirety.

Any cyclodextrin compound having antiviral activity may be used in the compositions of the present invention. In particular, cyclodextrins comprising six to twelve glucose units can be used in the invention. In preferred embodiments, cyclodextrins used in the inventive compositions comprise BCD, or salts or derivatives thereof. In further embodiment, the cyclodextrins used in the invention can comprise α-cyclodextrin (ACD), or salts or derivatives thereof, or γ-cyclodextrin (GCD), or salts or derivatives thereof. Still further, the cyclodextrins used in the invention can comprise various combinations of one or more BCD, ACD, or GCD (or salts or derivatives thereof).

In addition to unsubstituted cyclodextrins, the compositions of the invention can include one or more cyclodextrin derivatives. As used herein, a cyclodextrin derivative refers to a cyclodextrin wherein one or more of the hydroxyl groups have been altered through chemical reaction to introduce one or more different chemical moieties into the cyclodextrin molecule. Since each cyclodextrin hydroxyl group differs in its chemical reactivity, the reaction process in preparing a derivative forms an amorphous mixture of thousands of positional and optical isomers. Theoretically a cyclodextrin molecule (alpha, beta, or gamma) can have up to 3(n) substituents, where n is the number of glucopyranose units comprising the cyclodextrin molecule. This is referred to as the degree of substitution (DS). The DS refers to substituents other than hydrogen, and substituents may be all of one kind or a mixture of substituents. Cyclodextrins useful in the invention can have varying degrees of substitution, typically up to 100% substituted. In certain embodiments, the cyclodextrins may have a lesser mean DS, such as in the range of about 1.5 to about 2.1 substituent units per glucose molecule (i.e., approximately 25%-33% substituted).

Cyclodextrin derivatives used according to the invention can comprise cyclodextrin molecules wherein one or more of the OH groups are replaced with an OR group, wherein R comprises a variety of substituting groups. In specific embodiments, R comprises straight or branched alkyl, alkenyl, or alkynyl groups optionally substituted with one or more substituents, such as amino, amido, hydroxyl, ether, ester, or sulfonyl. In preferred embodiments, the alkyl, alkenyl, and alkynyl groups comprise 1-8 carbon atoms, 1-6 carbon atoms, or 1-4 carbon atoms. Specific, non-limiting examples of substituents that may be present on cyclodextrins useful in the invention include methyl, ethyl, propyl, butyl, hydroxymethyl, hydroxy ethyl, hydroxypropyl, hydroxybutyl, and sulfobutyl ether. Further examples of cyclodextrin derivatives useful according to the invention are described in U.S. Pat. No. 4,727,064, U.S. Pat. No. 5,376,645, and U.S. Pat. No. 6,001,343, all of which are incorporated herein by reference in their entirety. In one preferred embodiment, the compositions of the invention comprise hydroxypropyl BCD.

In one particular embodiment, the cyclodextrin used according to the invention comprises hydroxypropyl BCD (β-cyclodextrin, 2-hydroxypropyl ether). Such a cyclodextrin is freely water soluble and can have a DS less than 100%, such as in the range of about 0.4 to about 1.5 substituent units per glucose molecule. Of course, further cyclodextrin derivatives, or combinations thereof, could be used according to the invention, particularly derivatives that show antiviral activity.

As previously noted, cyclodextrins are well known for their use in combination with active agents to increase the solubility of the active agent or to other improve the physical properties of the formulation. According to the present invention, cyclodextrins themselves are used as active agents to treat or prevent viral infections. Such antiviral activity of cyclodextrins is more fully described below.

The present invention may thus be characterized in that the formulations comprise cyclodextrins as the lone active agent. Accordingly, the formulations of the present invention can be described as specifically excluding any further antiviral agents. In a broader sense, the formulations of the present invention can be described as specifically excluding any further active agents at all. In particular, the formulations can be described as comprising a cyclodextrin, wherein the cyclodextrin is not in the form of an inclusion complex with another pharmaceutically active compound.

Of course, the present invention is not solely limited to formulations wherein cyclodextrins are the only active agent. Rather, as may be determined beneficial, the formulations of the invention can be optimized by inclusion of one or more further active agents, such as antibiotics, analgesics, antiseptics, antifungals, anti-inflammatories, and the like. Moreover, in certain embodiments, the formulations of the invention may include one or more further antiviral agents. When such further active agents are present, it is preferable that the further agents are specifically not in the form of an inclusion complex with the cyclodextrins.

### Anti-Viral Activity

In addition to their use as drug carrier and modifiers, some data indicate cyclodextrins, particularly β-cyclodextrin (or BCD) has activity against HIV-1 (see, for example, U.S. Pat. No. 6,835,717). More recent studies have indicated cyclodextrins have antiviral activity against other types of viruses as well. For example, U.S. Patent Application Publication 2003/0220294, which is incorporated herein in its entirety, discloses that BCD has a specific antiviral effect against multiple viruses, such as HSV, vaccinia, Epstein-Barr virus, and HCV. Accordingly, the compositions of the invention comprising one or more cyclodextrins, or salts or derivatives thereof, are effective for treating or preventing viral infections.

As used herein, “treating” refers to ameliorating at least one symptom of a viral infection, preventing worsening of an existing viral infection, preventing occurrence of further symptoms after presentation of one or more viral symptoms, or lessening the duration of an active breakout of a viral infection (whether a first occurrence or a recurring viral infection). The term “preventing” refers to attenuating or reducing the ability of a virus to cause infection or disease, such as by affecting a post-entry viral event. For example, “preventing” can refer to attenuating the primary infection or transmission of the virus.

HSV

Over 40 million Americans suffer from cold sores (a common form of oral herpes) which are caused by HSV-1. HSV-1 can be transmitted through oral secretions, such as during kissing or by using contaminated food preparations and utensils. HSV-1 is also linked to approximately 5-10% of genital herpes infections. Initial oral herpes infections with HSV-1 typically occur in childhood (such as through casual contact with an infected individual), and thus are not classified as STDs.

HSV-2 causes the majority of genital herpes cases, one of the fastest growing STDs in the world. Roughly 86 million people worldwide are infected with HSV-2, of which 22 million display symptoms of painful genital blisters and sores with typically 5 to 8 outbreaks annually. Only about 2.6% of those afflicted with genital herpes have symptomatic infections. HSV-2 can be transmitted through direct personal contact and/or through oral or genital secretions, regardless of the presence of the symptoms.

Primary herpes virus infection occurs through a break in the mucous membranes of the mouth or throat, via the eye or genitals, or directly via minor abrasions in the skin. Because of the global distribution of HSV-1, most individuals are infected by 1-2 years of age. Initial infection is usually asymptomatic, although there may be minor local vesicular lesions. Local multiplication ensues, followed by viremia and systemic infection. A life-long latent infection with periodic reactivation generally follows.

During an initial (primary) infection, the herpes virus enters peripheral sensory nerves and migrates along axons to sensory nerve ganglia in the central nervous system (CNS), thus escaping an immune response. During latent infection of nerve cells, viral DNA is maintained as an episome (i.e., it is not integrated). There is, however, limited expression of specific virus genes required for the maintenance of latency. Outbreaks are triggered by various disturbances, such as physical trauma, ultraviolet light, hormones, stress, surgical trauma, or psychological trauma, which affect the immune system or hormonal balance.

Reactivation of latent virus leads to recurrent episodes of the disease. During recurrent infections, virus is reactivated and travels down sensory nerve ganglia to the surface of the body, re-infecting the skin and replicating, which causes tissue damage. Although painful, most recurrent infections resolve spontaneously, usually to reoccur later. More serious conditions include herpetic keratitis (ulceration of cornea due to repeated infections that can lead to blindness) and encephalitis, which is very rare and often fatal. Genital herpes is usually transmitted sexually and hence its incidence can be reduced or eliminated by use of appropriate vaginal anti-viral agents.

Epstein-Barr virus, frequently referred to as EBV, is another member of the herpes virus family and one of the most common human viruses. The virus occurs worldwide, and most people become infected with EBV sometime during their lives. When infection with EBV occurs during adolescence or young adulthood, it causes infectious mononucleosis 35-50% of the time. Symptoms of infectious mononucleosis are fever, sore throat, and swollen lymph glands. Sometimes, a swollen spleen or liver involvement may develop. Heart problems or involvement of the central nervous system can occur. EBV also establishes a lifelong dormant infection in some cells of the body's immune system. A late event in a very few carriers of this virus is the emergence of Burkitt's lymphoma and nasopharyngeal carcinoma. EBV appears to play an important role in these malignancies, but is probably not the sole cause of disease. Currently, there are no specific treatment options available for infectious mononucleosis, other than treating the symptoms.

Human Papillomavirus

In recent years, HPVs have been shown to be a group of the most common sexually transmitted viruses in the U.S. Up to 20 million Americans are currently infected with sexually transmitted HPVs, which are double stranded DNA viruses that cause genital warts (condylomata acuminata). It is estimated that about 75 percent of adult population has been infected with genital HPV at some point in their lives. While there are more than 65 types of HPV, over 90% of cases of genital warts are due to HPV types 6 and 11, but infection with specific types of HPV (mainly types 16, 18, 31, and 45) can lead to neoplastic changes in genital epithelia resulting in cancers of lower genital tract, including commonly occurring cervical carcinomas of women. Moreover, scientists have found association between several types of HPV and development of a number of cancers, including oral cancer and cancers of the anogenital region, such as cervical, vulvar, anal, and penile cancer. Because of the contagious spread and carcinogenic potential, HPV infections require treatment.

Depending on factors such as their size and location, genital warts are treated in several ways. Undiluted trichloroacetic acid preparation (TCA) can be applied to the infected area and washed off several hours later. An alternative treatment is a 20% podophyllin solution, which is applied to the affected area and later washed off. Pregnant women should not use podophyllin because it is absorbed by the skin and may cause birth defects in babies. Applications of 5% 5-fluorouracil cream may also be prescribed, although, as with podophyllin, it should be avoided during pregnancy. In addition, small warts can be removed by destructive methods, such as cryosurgery (freezing) or electrocautery (burning). Surgery is occasionally needed to remove large warts that have not responded to other treatment. Side effects that may occur with conventional treatments include pain, burning, inflammation, skin erosion, scarring, and erythema.

The drug alpha interferon is used when warts have recurred after removal by traditional means. In studies supported by NIAID and others, investigators found that interferon treatment eliminated the warts in about half of the patients. For some patients, though, a second course of treatment may be necessary. Although these treatments can eliminate the warts, they do not cure the disease, and warts often reappear after treatment.

Hepatitis Viruses

Hepatitis B is a sexually transmitted disease caused by the hepatitis B virus (HBV). Chronic infections can lead to severe liver damage (cirrhosis) and liver cancer (hepatocellular carcinoma). Hepatitis C is emerging as a serious liver disease, with a significantly higher risk for IV drug abusers and sexually promiscuous individuals. This disease is caused by the hepatitis C virus (HCV) which, unlike HBV, establishes chronic infections regardless of the age of infected persons and hence has a much higher potential to cause cirrhosis and hepatocellular carcinoma.

Human Cytomegaloviruses and Molluscum Contagiosum Virus

In addition to herpes virus, HPVs, and hepatitis viruses, which are capable of causing diseases in healthy individuals (primary pathogens), human cytomegaloviruses (HCMV) and molluscum contagiosum virus (MCV) are examples of further viruses capable of sexual transmission and usually causing opportunistic infections. In general, viruses like these become clinically significant when presented with other complications, usually in immunocompromised persons, for example, patients suffering from AIDS or other forms of immunodeficiencies, or patients on therapy for different types of transplantation or cancer.

HCMV causes one of the most common and difficult opportunistic infections in immunocompromised patients. The condition can result from primary infection, recurrence by the latent virus reactivation, or re-infection with a new strain of virus in otherwise previously infected persons. In such circumstances, diagnosis is hard to establish because besides demonstrating the presence of virus (lab detection of virus), its etiology has to be established for the given condition (i.e., if CMV is causing the pathology). HCMV is frequently involved in retinitis in the AIDS patients. In addition to the horizontal route, HCMV is the cause of the most frequent congenital infection in humans (vertical transmission), which can be asymptomatic or symptomatic, indicative of multiple organ involvement. In addition, individuals born with such infections commonly develop sensorineural deafness (CNS sequelae). HCMV is also considered as the leading cause of brain damage in children.

MCV is a poxvirus that causes dermal lesions (noninflamed skin papules) on various parts of the body, including the torso area in children and anogenital area in persons who engage in anogenital sex. A typical lesion consists of a localized mass of hypertrophied and hyperplastic epidermis extending down into the underlying dermis, but without breaking the basement membrane and projecting above the adjacent skin as a visible tumor. These lesions may last from 2 weeks to 2 years, and cropping may occur as a consequence of multiple simultaneous infections or by localized mechanical spread. MCV-caused lesions may be quite persistent and disfiguring in persons suffering from AIDS. Transmission of the virus is through direct contact and through body fluids.

Pox Virus

In addition to MCV, described herein, pox viruses amenable to treatment according to the invention include vaccinia, smallpox virus (variola), cowpox, monkey pox, pseudocowpox and Orf (contagious pustular dermatitis) virus. Orf has been placed in the genus Parapoxvirus of the poxviruses. Additional human pathogens among poxviruses include yabapox virus, tanapox virus, and molluscum contagiosum virus, which is described in more detail herein.

Poxviruses are large, brick-shaped viruses about 300 nm×200 nm. They have a double-stranded DNA genome (about 200 Kb) enclosed within a core that is flanked by two lateral bodies. The surface of the virus particle is covered with filamentous protein components. The entire particle is enclosed in an envelope derived from the host cell membranes. Laboratory diagnosis of pox viruses may be undertaken by electron microscopy of negatively stained vesicle fluid or lesion material. Some pox viruses can be cultured on the chorio-allantoic membrane of chick embryos, where they form pocks, and some can be isolated by cell-culture.

Vaccinia, which has been used for immunization against smallpox, is a genetically distinct type of pox virus which grows readily in a variety of hosts. In humans it causes a localized pustule with scar formation. In immuno-compromised persons or eczematous persons it sometimes caused a severe generalized vaccinia infection.

### Formulations

Various formulations are provided according to the invention that are useful in the treatment or prevention of viral infections including, but not limited to, the types of viral infections described herein. Although the formulations may be described in relation to specific embodiments, such embodiments are provided for the sake of clarity in the description of the invention and are not intended to limit the scope of the invention.

Non-Water Based Topical Gel Formulation

Topical gels are particularly useful for delivery of an antiviral agent to the site of an active breakout arising from a viral infection. For example, topical gels can be applied to cold sores around the mouth of a user, as well as further skin breakouts symptomatic of viral infection. Moreover, such gels can be applied prior to active breakouts to prevent formation of sores.

The topical gels of the present invention comprise one or more cyclodextrins, or salts or derivatives thereof, effective to treat or prevent a viral infection. Any of the cyclodextrins described herein can be used in the topical gels. Particularly useful are BCDs, especially hydroxypropyl BCD. The topical gels can comprise the cyclodextrins in concentrations of about 0.1% to about 50% by weight based on the overall weight of the gel composition. In specific embodiments, the cyclodextrins are present in the topical gels in concentrations of about 5% to about 40% by weight, about 10% to about 30% by weight, or about 15% to about 25% by weight, based on the overall weight of the gel composition. In one specific embodiment, the topical gel composition comprises about 20% by weight of one or more cyclodextrins, or salts or derivatives thereof.

The inventive topical gels are particularly characterized in that they are non-water based gels. As used herein, a “non-water based gel” is intended to mean a gel that is not based on an aqueous solvent. Gels typically are formed of a gelling agent (the dispersed phase) in an aqueous solvent (the continuous phase) to produce a viscous, jellylike product. For example, 2% gelatin in water is known to form a stiff gel. Such gels are typically made by cooling a solution of the gelling agent in water so that the gelling agent forms submicroscopic crystalline particle groups that retain much solvent in the interstices.

In the topical gels of the present invention, the non-water based gels are not formed of a gelling agent in a water solvent. Thus, while it is possible for the non-water based gels to include an amount of water, the water is present in an amount insufficient to act as a solvent or the overall composition of the gel is not such that the water can act as a solvent. As such, the water does not form a major component of the water-free gel. In such embodiments, the non-water based gels can be referred to as being substantially free of water. To be substantially free of water, the non-water based gels preferably comprise water in amounts less than about 25% by weight based on the overall weight of the gel composition. More preferably, the non-water based gels comprise less than about 20% by weight, less than about 15% by weight, less than about 10% by weight, less than about 5% by weight, less than about 4% by weight, less than about 3% by weight, less than about 2% by weight, less than about 1% by weight, less than about 0.5% by weight, or less than about 0.1% by weight, based on the overall weight of the gel composition.

The topical gel according to this embodiment of the invention is substantially free of water in that water is only included in the composition as being inherently present in one or more of the components of the composition. For example, components such as hydroxypropyl BCD may inherently contain a small amount of water that is carried over into the gel of the invention. Thus, water may be present in the topical gel, but the topical gel of the invention is substantially water free in that the water present in the gel is only included as a by-product of one or more of the component parts and does not play an actual role (such as a solvent) in the formation of the gel.

The non-water based topical gel of the invention is further characterized in that it can include little or no preservative. Typical water-based gels necessarily include preservatives to maintain shelf-life of the product. The topical gel of the present invention, being non-water based, can be prepared without the inclusion of preservatives, which reduces cost, simplifies the formulation, and eliminates the possibility of adverse reactions by users, which is not uncommon (e.g., the mercury-containing preservative thimerosal).

Water-based gels are disadvantageous in certain applications because they are free whisked away by bodily fluids (such as saliva) or external aqueous fluids (e.g., mouth medications solubilized by beverages). Non-water based gels, such as those described herein, are beneficial in that they remain on the application site longer and thus allow for prolonged activity at the site of application by the active component, such as the cyclodextrins of the present invention. Bioadhesive gels are described in U.S. Pat. No. 5,192,802, U.S. Pat. No. 5,314,915, and U.S. Pat. No. 5,298,258, all of which are incorporated herein by reference in their entirety, and such bioadhesive gels could be used with the cyclodextrins described herein to prepare topical gels according to the present invention.

The non-water based gels of the present invention are particularly beneficial in that they provide an occlusive effect. As pointed out above, water-based gels are plagued by the evaporative effect (i.e., the water solvent evaporates quickly allowing the remaining components to be easily whisked away). In the non-water based gels of the present invention, however, the absence of the water solvent significantly reduces or completely eliminates the evaporative effect. Rather, an occlusive effect is observed, and the active components of the gel are held in contact with the skin for a prolonged period of time to increase the effectiveness of the gel.

The occlusive effect is particularly beneficial with the use of relatively large molecules, such as cyclodextrins. For example, HPBCD has a molecular size that is normally too large to enter the human body through the skin (e.g., the skin penetration of HPBCD alone is typically in the range of about 0.1%). Therefore, in a water-based gel, where the evaporative effect is seen, components such as HPBCD have little time to interact with the skin and the effective skin penetration of the HPBCD is low. In the present non-water based gels, where the occlusive effect is seen, skin penetration of the cyclodextrin component can be increased on the order of 10 to 100 times. Thus, a significantly greater amount of the active component is allowed to interact with the skin and the beneficial aspects of the compositions, such as the anti-viral effect, are greatly increased.

In addition to the cyclodextrin component, the topical gels of the invention can comprise further gel components. For example, the topical gels can contain one or more non-aqueous solvents. Non-limiting examples of non-aqueous solvents useful according to the invention include lower alkyl alcohols (particularly C₁-C₆ alcohols), pyrrolidones, and volatile silicones. Specific examples of solvents useful according to the invention include, but are not limited to, methanol, ethanol, isopropyl alcohol, ethoxydiglycol, 1-methyl-2-pyrrolidone, polydimethylsiloxane, polyorganosiloxanes, and other silicone polymers.

In further embodiments, the non-water based topical gels can include one or more polyethylene glycol (PEG) components. As known in the art, PEG polymers are polymers according to the general structure shown below in formula (II)

—CH₂CH₂O—(CH₂CH₂O)ₙ—CH₂CH₂— (II)

wherein n is an integer from about 10 to about 4,000. Any PEG polymer according to the above structure could be useful according to the invention. In one particular embodiment of the invention, n can be an integer from about 50 to about 3,000, more particularly about 100 to about 2,000, still more particularly about 200 to about 500. In one specific embodiment of the invention, n is an integer from about 250 to about 450, particularly about 300 to about 400.

PEG is a highly versatile polymer available in multiple forms, making it particularly useful according to the present invention. The PEG component, for example, can exist in its non-bound form as a linear polymer with terminal hydroxyl groups as shown below in formula (III)

HO—CH₂CH₂O—(CH₂CH₂O)ₙ—CH₂CH₂—OH (III)

which can be abbreviated as HO-PEG-OH, wherein the PEG portion is understood to represent the structure provided above in formula (II). Multi-arm or branched PEG polymers are also useful according to the present invention. Multi-arm PEG polymers generally have two or more PEG backbones extending from a non-reactive linking chain.

As described above in relation to Formula (II), the molecular weight of the PEG polymers can vary depending upon the value of n. PEG polymers of varying molecular weight can be used according to the invention. For example, in one embodiment, the PEG component can have a molecular weight of up to about 100,000 Da, up to about 50,000 Da, up to about 20,000 Da, up to about 10,000 Da, up to about 5,000 Da, up to about 2,000 Da, or up to about 1,000 Da. In certain embodiment, the PEG component has a molecular weight in the range of about 200 Da to about 10,000 Da, about 200 Da to about 8,000 Da, about 200 Da to about 4,000 Da, about 500 Da to about 4,000 Da, or about 1,000 Da to about 3,000 Da. Unless otherwise noted, molecular weight is expressed herein as weight average molecular weight (M_{w}), which is defined by formula (IV) below

$${M}_{w} = \frac{\sum {n}_{i} {M}_{i}^{2}}{\sum {n}_{i} {M}_{i}} , \qquad ( IV )$$

wherein nᵢ is the number of polymer molecules (or the number of moles of those molecules) having molecular weight Mᵢ.

The non-water based gels of the invention can comprise one PEG component or a combination of two or more PEG components useful to achieve a desired gel viscosity. For example, the composition can comprise a single PEG component having a nominal viscosity generally in the range desired for the final viscosity of the non-water based gel product. In another embodiment, the composition can comprise two PEG components (e.g., a higher viscosity PEG and a lower viscosity PEG) in appropriate ratios such that the combination of the PEG components results in a desired final viscosity. Preferably, the final gel composition has a viscosity in the range of about 10 cP to about 500 cP, about 20 cP to about 400 cP, about 30 cP to about 300 cP, about 40 cP to about 250 cP, about 50 cP to about 200 cP, or about 75 cP to about 150 cP.

In certain embodiments, the non-water based gel of the invention can comprise up to about 99.9% by weight of one or more PEG components. Preferably, the gel composition comprises about 50% to about 99%, about 55% to about 95%, about 60% to about 90%, or about 75% to about 85% by weight of one or more PEG components, based on the overall weight of the gel composition. In specific embodiments, the non-water based gel composition consists of one or more cyclodextrin components and one or more PEG components.

In still further embodiments, the non-water based topical gel of the invention can include additional components including, but not limited to, oils, fats, thickeners, solubilizers, acids, and bases. Non-limiting examples of further additives include polycarbophil, polyacrylic acid, polyacrylates, polyvinylpyrrolidone, and alkyl celluloses (such as methyl cellulose, ethyl cellulose, propyl cellulose, or butyl cellulose).

In further preferred embodiments, the non-water based gels can comprise one or more paraffins. Such paraffins can be used in combination with one or more PEG components. In further embodiments, the gels can use paraffins as an alternative to PEG components. Any paraffin typically used in the preparation of hydrophobic or lipophilic gels can be used according to the invention.

Spray Formulation

In another aspect, the invention provides compositions in the form of spray formulations. Such formulations are particularly useful for delivery of the cyclodextrin composition to the areas of the body wherein application of a topical gel may not be convenient or as effective. In particular embodiments, the spray formulation is in the form of a nose spray or a throat spray. A nose spray is understood to refer to a spray composition amenable to spraying into one or both nostrils of a mammal and safe for contact with the mucous membranes within the nasal passages. A throat spray is understood to refer to a spray composition amenable to spraying into the mouth of a mammal. Particularly, the throat spray is intended to predominately by-pass the mouth of the user (i.e., the majority of the spray does not necessarily contact the tongue, palate, or interior cheek surfaces) and be applied to the throat area generally. Of course, the throat spray should be safe for contact with all surfaces of the mouth and throat. The spray formulations, of course, are not limited to use in the mouth or nose and could be used for application to other parts of the body as well.

The spray formulations of the present invention comprise one or more cyclodextrins, or salts or derivatives thereof, effective to treat or prevent a viral infection. Any of the cyclodextrins described herein can be used in the spray formulations. Particularly useful are BCDs, especially hydroxypropyl BCD. The spray formulations can comprise the cyclodextrins in concentrations of about 0.1% to about 50% by weight based on the overall weight of the spray composition. In specific embodiments, the cyclodextrins are present in the spray formulations in concentrations of about 1% to about 40% by weight, about 1% to about 30% by weight, about 1% to about 20% by weight, about 1% to about 10% by weight, about 1% to about 9% by weight, about 2% to about 8%, about 3% to about 7%, or about 4% to about 6% by weight, based on the overall weight of the spray formulations. In one specific embodiment, the spray formulation comprises about 5% by weight of one or more cyclodextrins, or salts or derivatives thereof.

The spray formulations of the invention can further comprise other components useful for preparing a formulation amenable to nose or throat application. For example, the spray formulations can include carriers, penetration enhancers, acids, bases, flavorants, and the like.

In certain embodiments, the spray formulations include one or more polyols. As used herein, a polyol is intended to refer to any compound containing multiple hydroxyl groups. More particularly, polyols can refer to polymers or monomers with hydroxyl functional groups available for organic reactions. Polymeric polyols may be polyethers such as polyethylene glycol or polypropylene glycol. Specific examples of polyols useful according to the present invention include ethylene glycol, propylene glycol, glycerin, pentaerythritol, 1,2-propanediol, dimethylpolysilanol, monomethyl ether, monoethyl ether, monobutyl ether, and diethylene glycol. The one or more polyols can be included in the spray formulation in concentrations in the range of 0.1% to about 30% by weight, about 1% to about 30% by weight, about 5% to about 30% by weight, or about 5% to about 20% by weight.

The spray formulations of the invention are preferably water-based formulations, which is intended to mean that the formulations include at least one aqueous solvent. Preferably, the spray formulations comprise water as the major solvent. Other solvents, however, may also be used. For example, the formulations may include further solvents, such as alcohols. Polyols, such as glycerol, are particularly useful in light of their water-binding effect.

Typically, water comprises up to about 99% by weight of the spray formulation based on the overall weight of the formulation. In certain embodiments, water comprises up to about 95%, up to about 90%, up to about 80%, up to about 70%, or up to about 60% by weight of the spray formulation. In further embodiments, water comprises about 50% to about 95% by weight of the spray formulation, preferably about 60% to about 90% or about 70% to about 90%.

Hydrogel Formulation

In another aspect, the invention provides compositions in the form of gels that are particularly suited for use in the nose or nasal passages of a mammal. Such gels can be non-water based gels, such as described above. In further embodiments, the nose gels of the invention are water-based gels.

The hydrogel formulations of the present invention comprise one or more cyclodextrins, or salts or derivatives thereof, effective to treat or prevent a viral infection. Any of the cyclodextrins described herein can be used in the hydrogel formulations. Particularly useful are BCDs, especially hydroxypropyl BCD. The hydrogel formulations can comprise the cyclodextrins in concentrations in the range of about 0.1% to about 50% by weight based on the overall weight of the hydrogel formulations. In specific embodiments, the cyclodextrins are present in the nose gel formulations in concentrations of about 1% to about 40% by weight, about 1% to about 30% by weight, about 1% to about 20% by weight, about 1% to about 10% by weight, about 2% to about 8%, about 3% to about 7%, or about 4% to about 6% by weight, based on the overall weight of the hydrogel formulations. In one specific embodiment, the hydrogel formulation comprises about 5% by weight of one or more cyclodextrins, or salts or derivatives thereof.

The hydrogel formulations of the present invention distinctly differ from the non-water based gels in that the hydrogel formulations are water-based gels. Accordingly, in specific embodiments, the hydrogel formulations of the invention comprise one or more carriers or solvents in combination with one or more thickeners or gel forming agents. Any solvent useful in the formation of gels (including aqueous solvents) may be used according to the invention. Gel forming agents useful according to the invention include, but are not limited to, cellulose ethers, such as methyl cellulose, hydroxyethyl cellulose, or carboxymethyl cellulose, and vegetable hydrocolloids, such as sodium alginate, tragacanth, or gum arabic.

In certain embodiments, the hydrogel formulations further comprise one or more co-solvents. In preferred embodiments, the co-solvents comprise one or more polyols. Non-limiting examples of polyols useful as co-solvents according to the present invention include Specific examples of polyols useful according to the present invention include ethylene glycol, propylene glycol, glycerin, pentaerythritol, 1,2-propanediol, dimethylpolysilanol, monomethyl ether, monoethyl ether, monobutyl ether, and diethylene glycol. The one or more polyols can be included in the spray formulation in concentrations in the range of 0.1% to about 30% by weight, about 1% to about 30% by weight, about 5% to about 30% by weight, or about 5% to about 20% by weight.

The hydrogels of the present invention can further comprise one or more components useful for making the hydrogels more compatible and non-irritating to the mucus membranes of the nasal passages. In such embodiments, the invention particularly provides nose gels. In preferred embodiments, the nose gels include one or more electrolytes useful for increasing the salinity of the nose gel. Bodily fluids, including those bathing the mucous membranes of the nasal passages, have a specific electrolyte balance and altering such electrolyte balance can cause irritating effects. Thus, the nose gels of the invention preferably include one or more salt components in concentrations useful to maintain the natural electrolyte balance of the mucous membranes within the nasal passages. In certain embodiments, the nose gel compositions comprise sodium chloride. Such additional components are not, however, limited to specific nose gel formulations and could be included in various further hydrogels according to the invention.

The hydrogel formulations can also comprise further components useful for delivery of the cyclodextrin component. For examples, the formulations can comprise penetration enhancers, humectants, emulsifiers, oils, fats, and other like components.

Balm Formulations

In another aspect, the invention provides compositions in the form of balms that are particularly suited for use on or around the lips of a mammal. In more specific embodiments, the balm formulations of the invention can comprise lipbalms, lipsticks, and other stick forms of balms. The stick balms can particularly be cosmetics, such as colored lipsticks or lip glosses. In further embodiments, the stick balms can be essentially colorless and may or may not include a scent or flavoring agent.

The lip balm formulations of the present invention comprise one or more cyclodextrins, or salts or derivatives thereof, effective to treat or prevent a viral infection.

Any of the cyclodextrins described herein can be used in the lip balm formulations. Particularly useful are BCDs, especially hydroxypropyl BCD. The lip balm formulations can comprise the cyclodextrins in concentrations of about 0.1% to about 50% by weight based on the overall weight of the lip balm formulations. In specific embodiments, the cyclodextrins are present in the lip balm formulations in concentrations of about 5% to about 50% by weight, about 5% to about 40% by weight, about 10% to about 40%, about 10% to about 30%, or about 15% to about 25% by weight, based on the overall weight of the lip balm formulations. In one specific embodiment, the lip balm formulation comprises about 20% by weight of one or more cyclodextrins, or salts or derivatives thereof.

The lip balm formulations of the invention generally comprise one or more base forming components that comprise the bulk of the balm. For example, solid sticks can comprise natural or synthetic waxes, fatty alcohols, or fatty acid esters as the base forming component. Specific examples of bases which are suitable for use as in the lip balms of the present invention are liquid oils (e.g., paraffin oils, castor oil, cetosearyl alcohol, and isopropyl myristate), semisolid constituents (e.g., Vaseline and lanolin), solid constituents (e.g., beeswax, ceresine and microcrystalline waxes and ozokerite), and high-melting waxes (e.g., carnauba wax and candelilla wax). All of the foregoing base forming components can be described, as a group, by the phrase “waxy components”. Thus, as used in relation to a lip balm according to the invention, a waxy component is any of the noted materials that can be used to form the bulk of the lip balm and, like waxes, are generally solid or semi-solid at ambient temperature but are at least softened at temperatures approaching the average human body temperature (i.e., about 37° C.).

In preferred embodiments, the compositions of the invention further comprise one or more sunscreen components. Particularly, the sunscreens can comprise at least one UVA filter substance and/or at least one UVB filter substance and/or at least one inorganic pigment. Many viral infections, particularly recurring infections, are susceptible to UV-stimulated breakouts (i.e., breakouts stimulated by exposure to sunlight). Accordingly, addition of UV filters to the compositions of the invention can be particularly useful for reducing or preventing breakouts.

The UVB filters used according to the invention can be oil-soluble or water-soluble. Examples of oil-soluble substances are: 3-benzylidenecamphor and derivatives thereof, e.g. 3-(4-methylbenzylidene)camphor; 4-aminobenzoic acid derivatives, preferably 2-ethylhexyl 4-(dimethylamino)benzoate, amyl 4-(dimethylamino)benzoate; esters of cinnamic acid, preferably 2-ethylhexyl 4-methoxycinnamate, isopentyl 4-methoxycinnamate; esters of salicylic acid, preferably 2-ethylhexyl salicylate, 4-isopropylbenzyl salicylate, homomethyl salicylate; derivatives of benzophenone, preferably 2-hydroxy-4-methoxybenzophenone, 2-hydroxy-4-methoxy-4′-methylbenzophenone, 2,2′-dihydroxy-4-methoxybenzophenone; esters of benzalmalonic acid, preferably di(2-ethylhexyl) 4-methoxybenzalmalonate; and 2,4,6-trianilino(p-carbo-2′-ethyl-1′-hexyloxy)-1,3,5-triazine. Advantageous water-soluble substances are: 2-phenylbenzimidazole-5-sulfonic acid and salts thereof, e.g., sodium, potassium or triethanolammonium salts; sulfonic acid derivatives of benzophenones, preferably 2-hydroxy-4-methoxybenzophenone-5-sulfonic acid and its salts; and sulfonic acid derivatives of 3-benzylidenecamphor, such as, for example, 4-(2-oxo-3-bornylidenemethyl) benzenesulfonic acid, 2-methyl-5-(2-oxo-3-bornylidenemethyl)sulfonic acid and its salts. The list of said UVB filters which can be used according to the invention is of course not intended to be limiting.

Examples of UVA filter substances useful according to the invention include derivatives of dibenzoylmethane, in particular 1-(4′-tert-butylphenyl)-3-(4′-methoxyphenyl)propane-1,3-dione and 1-phenyl-3-(4′-isopropylphenyl)propane-1,3-dione. Further examples of UV filters useful according to the invention include p-aminobenzoic acid, its salts and its derivatives (ethyl, isobutyl, glyceryl esters; p-dimethylaminobenzoic acid); anthranilates (o-aminobenzoates; methyl, menthyl, phenyl, benzyl, phenylethyl, linalyl, terpinyl, and cyclohexenyl esters); salicylates (octyl, amyl, phenyl, benzyl, menthyl (homosalate), glyceryl, and dipropyleneglycol esters); cinnamic acid derivatives (menthyl and benzyl esters, alpha-phenyl cinnamonitrile; butyl cinnamoyl pyruvate); dihydroxycinnamic acid derivatives (umbelliferone, methylumbelliferone, methylaceto-umbelliferone); camphor derivatives (3-benzylidene, 4-methylbenzylidene, polyacrylamidomethyl benzylidene, benzalkonium methosulfate, benzylidene camphor sulfonic acid, and terephthalylidene dicamphor sulfonic acid); trihydroxycinnamic acid derivatives (esculetin, methylesculetin, daphnetin, and the glucosides, esculin and daphnin); hydrocarbons (diphenylbutadiene, stilbene); dibenzalacetone and benzalacetophenone; naptholsulfonates (sodium salts of 2-naphthol-3,6-disulfonic and of 2-naphthol-6,8-disulfonic acids); dihydroxy-naphthoic acid and its salts; o- and p-hydroxydiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric and vilouric acids; tannic acid and its derivatives; hydroquinone; benzophenones (oxybenzone, sulisobenzone, dioxybenzone, benzoresorcinol, 2,2′,4,4′-tetrahydroxybenzophenone, 2,2′-dihydroxy-4,4′-dimethoxybenzophenone, octabenzone), dibenzoylmethane derivatives, avobenzone, 4-isopropyldibenzoylmethane, butylmethoxydibenzoylmethane, 4-isopropyl-dibenzoylmethane, octocrylene, and drometrizole trisiloxane

The lip balm formulations of the invention can further include sunscreens in the form of inorganic pigments which are customarily used in cosmetics for protecting the skin against UV rays. These can include oxides of titanium, zinc, iron, zirconium, silicon, manganese, aluminum, cerium, and mixtures thereof, and modifications in which the oxides are the active agents.

The lip balms of the invention can further comprise additional components. For example, the lip balms can include one or more antioxidants. The antioxidants are advantageously chosen from the group consisting of amino acids (for example glycine, histidine, tyrosine, tryptophan) and derivatives thereof, imidazoles (for example urocanic acid) and derivatives thereof, peptides, such as D,L-carnosine, D-carnosine, L-carnosine and derivatives thereof (for example anserine), carotenoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulphoximine compounds (for example buthionine-sulphoximines, homocysteine-sulphoximine buthionine sulphones, penta-, hexa- and heptathionine-sulphoximine) in very low tolerated doses (for example pmol to μmol/kg), and furthermore (metal) chelating agents (for example α-hydroxy fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, vitamin C and derivatives (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (for example ZnO, ZnSO₄), selenium and derivatives thereof (for example selenium methionine), stilbenes and derivatives thereof (for example stilbene oxide, trans-stilbene oxide) and the derivatives of these active ingredients mentioned which are suitable according to the invention (salts, esters, ethers, sugars, nucleotides, nucleosides, peptides and lipids).

Examples of other components that may be included in the lip balms include pigments and other coloring agents, flavorants, anti-inflammatories, essential oils, moisturizers, preservatives, and any other cosmetically safe components that may be useful.

Lipophilic and Hydrophilic Emulsion Formulations

In further embodiments, the invention provide additional formulations wherein the coponents are in the form of an emulsion. Such further formulations can be lipophilic in nature (i.e., fat based) or hydrophilic in nature (i.e., aqueous based) and can take on various specific forms (e.g., emulsion-based creams, lotion, and the like). The emulsions used in preparing the lipophilic and hydrophilic formulations include oil-in-water (O/W) emulsions, water-in-oil (W/O) emulsions, water-in-oil-in-water (W/O/W) emulsions, oil-in-water-in-oil (O/W/O) emulsions, lipodispersions, or hydrodispersions. Emulsions in the sense of the present invention, are advantageous and contain, for example, fats, oils, waxes, or other fat bodies, as well as water and one or more emulsifiers, as are typically used for such a type of formulation.

The lipid phase of an emulsion according to the invention can advantageously be chosen from the following group of substances: mineral oils and mineral waxes; oils, such as triglycerides of capric acid, caprylic acid, or castor oil; fats, waxes, and other natural and synthetic fatty bodies, preferably esters of fatty acids with alcohols of low carbon number, e.g., with isopropanol, propylene glycol or glycerol, or esters of fatty alcohols with alkanoic acids of low carbon number or with fatty acids; alkyl benzoate; silicone oils, such as dimethylpolysiloxanes, diethylpolysiloxanes, diphenylpolysiloxanes, and mixed forms thereof. Non-limiting examples from which the oil phase of the emulsions can be chosen include esters of saturated and/or unsaturated, branched and/or unbranched alkanecarboxylic acids with a chain length of from 3 to 30 carbon atoms and saturated and/or unsaturated, branched and/or unbranched alcohols with a chain length of from 3 to 30 carbon atoms; esters of aromatic carboxylic acids and saturated and/or unsaturated, branched and/or unbranched alcohols with a chain length of from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, e.g., jojoba oil. In addition, the oil phase can advantageously be chosen from the group of branched and unbranched hydrocarbons and hydrocarbon waxes, silicone oils, dialkyl ethers, the group of saturated or unsaturated, branched or unbranched alcohols, and fatty acid triglycerides, namely the triglycerol esters of saturated and/or unsaturated, branched and/or unbranched alkanecarboxylic acids with a chain length of from 8 to 24, in particular 12-18, carbon atoms. The fatty acid triglycerides can, for example, advantageously be chosen from the group of synthetic, semisynthetic and natural oils, e.g., olive oil, sunflower oil, soybean oil, peanut oil, rapeseed oil, almond oil, palm oil, coconut oil, palm kernel oil and the like.

Any mixtures of such oil and wax components can also be used advantageously for the purposes of the present invention. It may also in some circumstances be advantageous to use waxes, for example cetyl palmitate, as the sole lipid component of the oil phase. The oil phase can advantageously also have a content of cyclic or linear silicone oils or consist entirely of such oils, although it is preferred to use an additional content of other oil phase components apart from the silicone oil or the silicone oils.

The aqueous phase of the preparations according to the invention optionally advantageously comprises alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and also alcohols of low carbon number, e.g. ethanol, isopropanol, 1,2-propanediol, glycerol, and in particular one or more thickeners which can advantageously be chosen from the group consisting of silicon dioxide, aluminum silicates, polysaccharides and derivatives thereof, e.g. hyaluronic acid, xanthan gum, hydroxypropylmethylcellulose, particularly advantageously from the group of polyacrylates, preferably a polyacrylate from the group of so-called carbopols.

The creams, lotions, etc. prepared using the emulsions, hydrodispersions, or lipodispersions can include a variety of further components. For example, the compositions may further comprise UV-blockers or antioxidants, as described above, as well as preservatives, solubilizers, fragrances, conditioning agents, or moisturizers.

Many modifications and other embodiments of the inventions set forth herein will come to mind to one skilled in the art to which these inventions pertain having the benefit of the teachings presented in the foregoing descriptions. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A gel composition comprising:
a) about 0.1% to about 50% by weight of one or more cyclodextrins or salts or derivatives thereof; and
b) one or more polyethylene glycol-based components;
wherein said gel is a non water-based gel.

2. The gel composition of claim 1, wherein said composition is preservative-free.

3. The gel composition of claim 1, wherein said composition comprises less than about 10% by weight of water based on the overall weight of the composition.

4. The gel composition of claim 1, comprising about 10% to about 20% by weight of one or more cyclodextrins or salts or derivatives thereof.

5. The gel composition of claim 1, wherein said one or more cyclodextrins comprises one or more α-cyclodextrins.

6. The gel composition of claim 1, wherein said one or more cyclodextrins comprises one or more β-cyclodextrins.

7. The gel composition of claim 1, wherein said one or more cyclodextrins comprises one or more γ-cyclodextrins.

8. The gel composition of claim 1, wherein said composition specifically does not include any additional active agents.

9. A sprayable composition comprising:
(a) about 0.1% to about 50% by weight of one or more cyclodextrins or salts or derivatives thereof;
(b) one or more polyols; and
(c) water.

10. The sprayable composition of claim 9, comprising about 2.5% to about 7.5% by weight of one or more cyclodextrins or salts or derivatives thereof.

11. The sprayable composition of claim 9, wherein said one or more cyclodextrins comprises one or more α-cyclodextrins.

12. The sprayable composition of claim 9, wherein said one or more cyclodextrins comprises one or more β-cyclodextrins.

13. The sprayable composition of claim 9, wherein said one or more cyclodextrins comprises one or more γ-cyclodextrins.

14. The sprayable composition of claim 9, wherein said composition comprises a nose spray.

15. The sprayable composition of claim 9, wherein said composition comprises a throat spray.

16. The sprayable composition of claim 9, wherein said composition specifically does not include any additional active agents.

17. A nose gel composition comprising:
(a) about 0.1% to about 50% by weight of one or more cyclodextrins or salts or derivatives thereof;
(b) a carrier; and
(c) a thickener.

18. The nose gel composition of claim 17, comprising about 2.5% to about 7.5% by weight of one or more cyclodextrins or salts or derivatives thereof.

19. The nose gel composition of claim 17, wherein said one or more cyclodextrins comprises one or more α-cyclodextrins.

20. The nose gel composition of claim 17, wherein said one or more cyclodextrins comprises one or more β-cyclodextrins.

21. The nose gel composition of claim 17, wherein said one or more cyclodextrins comprises one or more γ-cyclodextrins.

22. The nose gel composition of claim 17, wherein said composition specifically does not include any additional active agents.

23. The nose gel composition of claim 17, further comprising one or more polyols.

24. The nose gel composition of claim 17, further comprising one or more electrolytes.

25. A lip balm composition comprising:
(a) about 0.1% to about 50% by weight of one or more cyclodextrins or salts or derivatives thereof; and
(b) one or more waxy components.

26. The lip balm composition of claim 25, comprising about 15% to about 25% by weight of one or more cyclodextrins or salts or derivatives thereof.

27. The lip balm composition of claim 25, further comprising one or more UV filter components.

28. The lip balm composition of claim 25, wherein said one or more cyclodextrins comprises one or more α-cyclodextrins.

29. The lip balm composition of claim 25, wherein said one or more cyclodextrins comprises one or more β-cyclodextrins.

30. The lip balm composition of claim 25, wherein said one or more cyclodextrins comprises one or more γ-cyclodextrins.

31. The lip balm composition of claim 25, wherein said composition specifically does not include any additional active agents.

32. An emulsified composition comprising about 0.1% to about 50% by weight of one or more cyclodextrins or salts or derivatives thereof, wherein said composition is in the form of a cream or lotion.

33. A pharmaceutical or cosmetic composition for application to a site where a primary viral infection actively exists or a site known to exhibit a recurrent viral infection, the composition comprising 0.1% to 50% by weight of a cyclodextrin or derivative thereof and a pharmaceutically or cosmetically acceptable excipient suitable such that the pharmaceutical or cosmetic composition is in the form of a non water-based gel, a nose or throat spray, a hydrogel suitable for application to mucus membranes, or a lip balm, wherein the pharmaceutically or cosmetically acceptable excipient is selected from the group consisting of polyethylene glycol-based components, polyols, electrolytes, and waxy components, and wherein the cyclodextrin is the only agent present in the composition having antiviral activity or is not in the form of an inclusion complex with a further compound having antiviral activity.
